(19) United States
(12) Patent Application Publication
(10) Pub. No.: US 2008/0219883 A1
(43) Pub. Date: Sep. 11, 2008

(54) TOOTHBRUSH SANITIZER

(76) Inventors: Charles Thur, Broadview Heights, OH (US); David Brickner, Concord, OH (US)

Correspondence Address: FAY SHARPE LLP, 1100 SUPERIOR AVENUE, SEVENTH FLOOR, CLEVELAND, OH, 44114, US

(21) Appl. No.: 12/044,408

(22) Filed: Mar. 7, 2008

Related U.S. Application Data
(60) Provisional application No. 60/894,051, filed on Mar. 9, 2007.

(51) Int. Cl. A61L 2/04

(52) U.S. Cl. 422/22; 422/307

(57) ABSTRACT

A toothbrush sanitizer includes at least one basket for holding a toothbrush, a housing that receives the at least one basket, and a heating element connected to the housing. The basket(s) can also accommodate a toothbrush head from a known electric toothbrush. The basket can be completely removable from the housing so that the basket can be separately cleaned from the housing. The heating element heats the toothbrush to sanitize the bristles. According to an alternative embodiment, the toothbrush sanitizer can include a first toothbrush basket, a second toothbrush basket, a housing that receives the baskets, and a heating element connected with the housing. The second basket receives the first basket.

This application claims the benefit of U.S. Application No. 60/894,051, filed Mar. 9, 2007, the entirety of which is incorporated by reference.

### BACKGROUND

Unsterile toothbrushes may be contributing factors to infections, as well as a carrier to the spread of diseases. Sterilization and sanitization systems typically employ a heating element and/or an anti-microbial spray for sterilizing the toothbrush bristles. These known devices typically include components that are difficult to clean, especially the components that come into contact with the toothbrush that is being sterilized or any water or other liquids that may be captured by the bristles of the toothbrush. Additionally, some of the known toothbrush sanitizers are unable to accommodate the many different sizes and shapes of toothbrushes and toothbrush heads that are available in the marketplace today.

### SUMMARY OF THE INVENTION

A toothbrush sanitizer that overcomes the aforementioned shortcomings includes at least one basket for holding a toothbrush, a housing that receives the at least one basket, and a heating element connected to the housing. The heating element heats the toothbrush to sanitize the bristles. The basket can also accommodate a toothbrush head from an electric toothbrush. The basket is completely removable from the housing so that the basket can be separately cleaned from the housing.

A method for sanitizing a toothbrush is also disclosed. The method includes positioning a toothbrush in a container so that the toothbrush is in a substantially vertical orientation with bristles of the toothbrush being situated highest, inserting the container into a housing that contains an electrically powered heating element, and operating a switch to deliver electricity to the heating element to heat the bristles of the toothbrush.

According to an alternative embodiment, a toothbrush sanitizer can include a first basket, a second basket, a housing that receives the baskets, and a heating element connected with the housing. The baskets are configured to accommodate toothbrushes and toothbrush heads. The second basket receives the first basket. The housing receives the baskets when the second basket is received in the first basket.

### BRIEF DESCRIPTION OF THE DRAWINGS

FIG. 1 is a front perspective view of a toothbrush sanitizer.

FIG. 2 is a rear perspective view of the toothbrush sanitizer shown in FIG. 1.

FIG. 3 is an exploded view of the toothbrush sanitizer shown in FIG. 1.

FIG. 4 is a side cross-sectional view of the toothbrush sanitizer shown in FIG. 1.

FIG. 5 is a rear perspective view of a first basket received in a second basket of the toothbrush sanitizer shown in FIG. 1.

FIG. 6 is a rear perspective view of the first basket removed from the second basket shown in FIG. 5.

FIG. 7 is a top plan view of the baskets shown in FIG. 5.

FIG. 8 is a rear elevation view, partially in cross section, of the baskets shown in FIG. 5.

### DETAILED DESCRIPTION

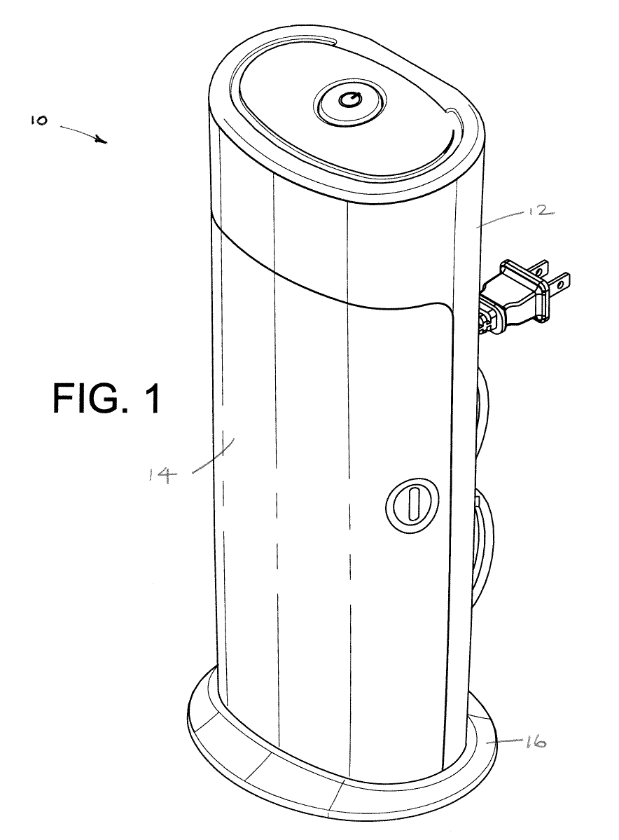

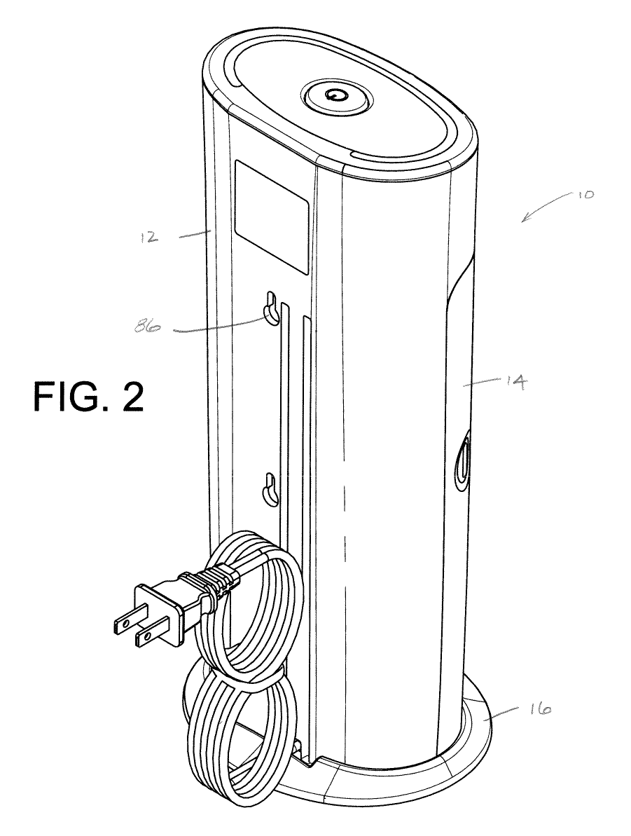

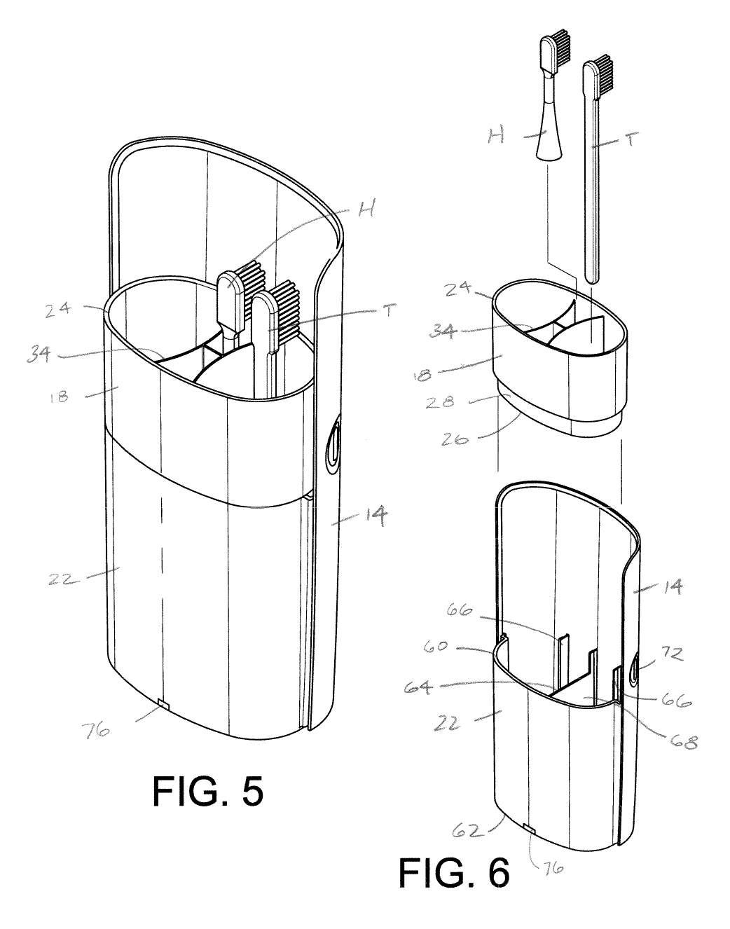

With reference to FIG. 1, the toothbrush sanitizer sanitizes and/or sterilizes toothbrushes T and H (FIGS. 5 and 6) using heat under ultraviolet light. The toothbrush sanitizer 10 includes a main housing 12, a front cover 14 and a base 16. Toothbrushes and toothbrush components, such as toothbrush heads used with an electric toothbrush, are positioned in baskets, which will be described in more detail below. The baskets are inserted into the housing. The bristles of the toothbrush or toothbrush heads are heated by a heating element, which will be described in more detail below, and are also subject to the photocatalytic properties of titanium dioxide under ultraviolet light.

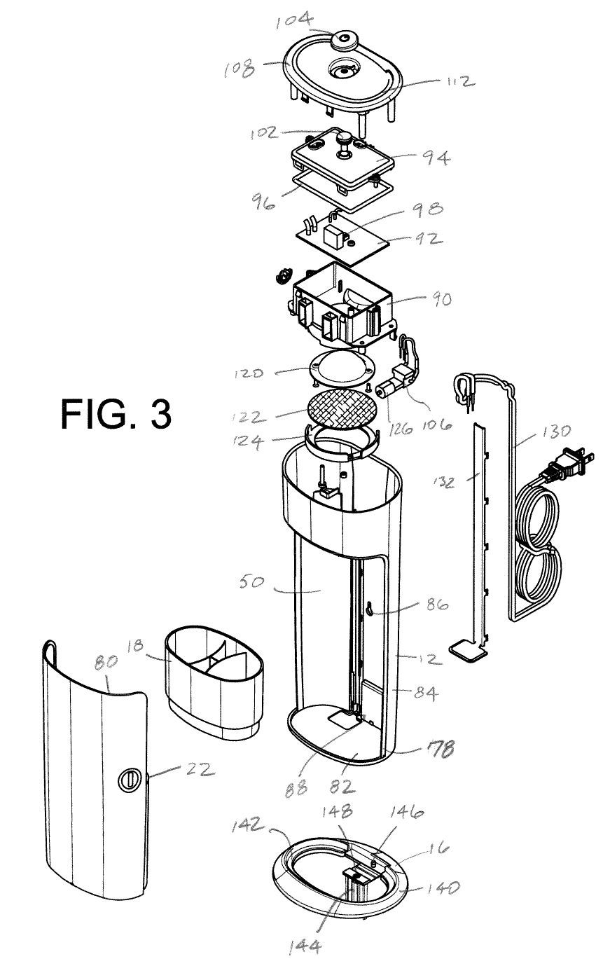

With reference to FIG. 3, the toothbrush sanitizer includes an upper (first) toothbrush basket 18 and a lower (second) toothbrush basket 22 (see also FIGS. 5 and 6). The housing 12 receives the baskets 18 and 22 and the baskets are completely removable from the housing so that the baskets can be separately cleaned from the housing. The baskets 18 and 22 (as well as the front cover 14, which is integrally formed with the lower basket 22) are made from a dishwasher-safe material.

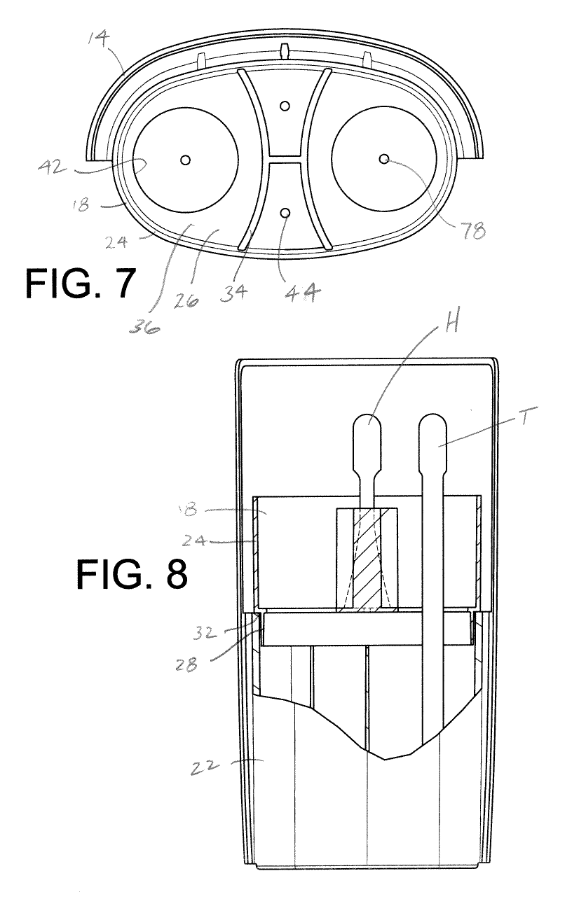

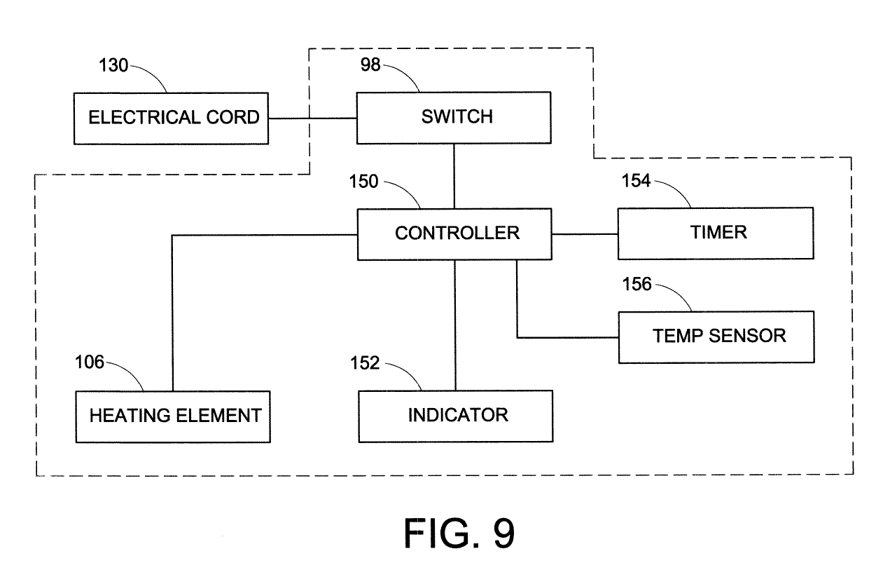

With reference to FIG. 5, the first basket 18 is received in and supported by the second basket 22. The upper basket 18 includes a continuous peripheral side wall 24 that extends upwardly from an integral base 26 (FIG. 6). A lower recessed portion 28 of the peripheral side wall 24 define a shoulder 32 (FIG. 8) that is supported by the lower basket 22. The upper basket 18 includes internal wails 34 that connect to the peripheral wall 24 to define a plurality of compartments 36. With reference to FIG. 7, at least one toothbrush opening 42 extends through the base 26. In the depicted embodiment, the base 38 of the upper basket 18 includes two openings 42 that are each formed in different compartments 36 and these openings each have a diameter large enough to allow the handle of a toothbrush, and more particularly a known electric toothbrush handle, to fit through the opening, e.g. a 1½ inch diameter. As seen in FIG. 7, the base also includes drain apertures 44 that have a diameter smaller than the diameter of the toothbrush openings 42. The drain apertures 44 allow water to drain from the upper basket 18.

With reference back to FIGS. 5 and 6, the second (lower) basket 22 is integrally formed with the front cover 14. The front cover 14 cooperates with the housing 12 to enclose an internal compartment 50 (FIG. 3) that completely encloses the toothbrush T (FIGS. 5, 6 and 8) and/or toothbrush head H (FIGS. 5, 6 and 8) that are received by the respective baskets. In the depicted embodiment, the lower toothbrush basket 22 is integrally formed with the front cover 14 so that the external surface of the front cover fits substantially flush with an outer surface of the housing 12 to enclose the internal compartment 50. The upper toothbrush basket 18 is disposed behind the front cover 14 and within the internal compartment 50 when the toothbrush baskets are received in the housing. In the depicted embodiment, the entire toothbrush T is enclosed, as opposed to only the bristles.

With reference to FIG. 6, the lower basket 22 is defined by the front panel 14, a rearwardly extending side wall 60, and a base wall 62. Both the front cover 14 and the rear wall 60 extend upwardly from the base wall 62. An internal wall 64 connects the front panel 14 to the rear wall 60. Spacers 66 extend rearwardly from the front cover 14. The distance between the rearward longitudinal edge of the spacers 66 and the rear wall 60 is sufficient to receive the recessed portion 28 of the upper basket 18. The internal wall 64 of the lower basket 22 and the side wall 60 support the base 26 of the upper basket 18 when the upper basket 18 is received in the lower basket 22 (see FIG. 8).

The internal wall 64 of the lower basket 22 in combination with the front cover 14 and the rear wall 60 define compartments 68 to receive toothbrushes T. Since toothbrush openings 42 are provided in the base wall 26 of the upper basket 18, the upper basket 18 can be received in the lower basket 22 and the handle of the toothbrush can extend through the toothbrush opening and rest on the base wall 62 of the lower basket (see FIG. 8). Indentions 72 are formed in the front cover 14 to provide a location for gripping the front cover to remove the front cover from the housing 12. Both the upper basket 18 and a lower basket 22 are completely removable from the housing 12 so that the toothbrush baskets can be separately cleaned from the housing. The toothbrush baskets are made from a material that can withstand heat of at least about 185° F. and are also made from a material, e.g. plastic, that is dishwasher safe. A magnet 76 is located at a lower edge of the rear wall 60, the significance of which will be described in more detail below. The lower basket 22 also includes drain apertures 78 (FIG. 7) formed through the base wall 62 similar to the drain apertures 44 found in the upper basket 18.

With reference back to FIG. 3, the housing 12 includes a recess 78 formed in a side wall 84 that receives a peripheral edge 80 of the front cover 14 so that an outer surface of the front cover 14 is substantially flush with an outer surface of the housing when the front cover is received by the housing (see FIG. 1). The housing 12 also includes a base wall 82 having substantially the same size and configuration as the base wall 62 of the front cover 14. The housing 12 is made from a material that is chemically compatible with paint, solvent, heat, and ultraviolet light. Examples of such materials are durable plastics and the like.

The side wall 84 of the housing 12 extends upwardly from the base 82. In the depicted embodiment, the side wall 84 extends upwardly from only a rearward portion of the periphery of the base 82 to generally define the enclosure 50 that receives the toothbrush baskets 18 and 22. The side wall 84 is generally curved in plan view to have a complementary shape to the toothbrush baskets that are disposed in the enclosure 50. Slotted openings 86 are provided in a rear planar section of the side wall 84 and are key-shaped to allow the toothbrush sanitizer 10 mount to fasteners, e.g. screws and nails, which are mounted on a wall. At an upper end of the housing 12, the side wall 84 completely surrounds the periphery of the base wail 82, although being vertically spaced from the base wall 82, to house electrical components of the toothbrush sanitizer. At a lower edge of the side wall 84 magnetic material 88 is provided that cooperates with the magnet 76 on the lower basket 22 to retain the lower basket in the enclosure 50.

With reference to FIG. 3, an electrical component chassis 90 connects with the housing 12. The electrical component chassis 90 receives a printed circuit board 92 that includes electrical components mounted on it which will be described in more detail below. A switch holder 94 connects to the electrical component chassis 90. A gasket 96 is interposed between the switch holder 94 and the electrical component chassis 90 to prevent the ingress of moisture and contaminants into the internal compartment of the electrical component chassis that could harm the printed circuit board 92. A switch 98 mounts to the printed circuit board 92. A movable plunger 102 moves to actuate the switch 98. A movable pushbutton 104 contacts the plunger 102 and is depressed by the user to deliver electrical energy to a heating element 106. A lid 108 connects to the housing 12 to cover the switch holder 94. Vents 112 are provided in the lid 108 to allow heat to escape the electrical component chassis and the upper compartment of the housing 12.

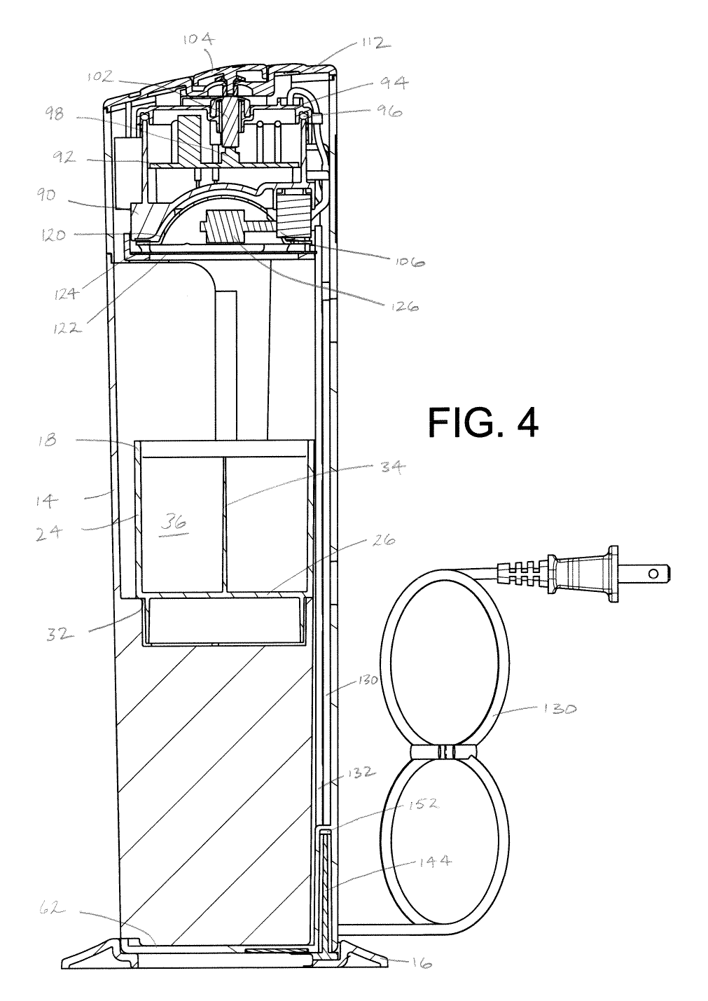

A reflector 120 connects to a lower surface of the electrical component chassis 90. A lower surface of the reflector 120 is coated with titanium dioxide. The heating element 106 also connects to the electrical component chassis 90 and is in electrical communication with the circuit board 94. A UV filter lens 122 that filters UVA and UVB radiation is disposed below the reflector 120 so that the heating element 106 is disposed in between the lens 122 and the reflector 120 (see FIG. 4). A retainer 124 retains the lens 122 to the reflector 120 and to the electrical component chassis 90.

The heating element 106 in the depicted embodiment is a 120 volt, 20 watt light bulb 126, which can be either xenon or halogen. The light bulb 126 can also be coated with titanium dioxide to help with the sanitization process. In lieu of using a light bulb 126, the heating element 106 can be a resistance-type heat source. Where the light bulb 126 is utilized, the reflector 120 is disposed adjacent the light source to direct radiation toward the toothbrushes and the toothbrush attachments. The light bulb 126 is installed in a socket that is known in the art. Accordingly, if the light bulb burns out, the light bulb can be easily replaced. The light bulb 126 is hidden to prevent a direct line of sight from outside the toothbrush sanitizer to the bulb.

In the depicted embodiment, the heating element 106 receives electrical energy from a wall outlet via an electrical cord 130. The electrical cord 130 connects to the PCB 92. An electrical cord cover 132 connects to the housing 12 to provide a channel in which the electrical cord resides. As is more clearly seen in FIG. 4, the electrical cord 130 is sandwiched between the electrical cord cover 132 and the housing 12.

The base 16 can connect to the housing 12 when the user of the toothbrush sanitizer 12 desires to rest the toothbrush sanitizer on a countertop or other surface. With reference to FIG. 3, the base 16 is an annular member 140 including a recess 142 that is configured to receive the base wall 82 of the housing 12. A key 144 is integrally formed with the annular member 140. The key 144 attaches to the annular member 140 via a living hinge. A post 146 extends upwardly from the ring-shaped member 140 to be received inside an opening 148 formed in the key 144. With reference to FIG. 4, the key 144 is received in a notch 152 formed in the housing for attaching the base 16 to the housing 12.

To use the toothbrush sanitizer 10 one inserts toothbrushes into the baskets 18 or 22 and then inserts the baskets into the enclosure 50 of the housing 12. The upper basket 18 need not be loaded into the housing 12 to operate the toothbrush sanitizer. Typically, smaller toothbrushes and toothbrush attachments are loaded into the upper basket 18 and more particularly in the compartments of the upper basket not having the large toothbrush openings 42. The toothbrushes and toothbrush attachments are loaded into the basket so that the toothbrush is in a substantially vertical orientation with the bristles being situated highest and the handle of the toothbrush resting on a respective base of a toothbrush basket 18 or 22. To accommodate the entire toothbrush T, the base wall 62 of the lower basket 22 is spaced below the lens 122, and thus the heating element 106, between about 9 inches and about 10 inches. To accommodate toothbrush attachments, e.g. heads H, and smaller (children's) toothbrushes the base wall 26 of the first basket 18 is spaced below the lens 122, and thus the heating element 106, between about 4.5 inches and about 5.5 inches.

The front cover 14, which includes the integrally formed lower basket 22, is then inserted into the housing 12 that contains the electrically powered heating element 106. The operator then operates the switch 98 by depressing the switch button 104 to deliver electricity to the heating element 106 to heat the bristles of the toothbrush. As explained above, a toothbrush head or a toothbrush can also be disposed in the upper basket 18 in a substantially vertical orientation with the bristles being situated highest and the upper basket can be inserted into the lower basket 22 prior to inserting the lower basket 22 into the housing 12.

FIG. 9 depicts a schematic depiction of the electrical components that can be found in the toothbrush sanitizer 10. The electrical components can include the heating element 106, a controller (control circuit) 150, an indicator 152, a timer 154 and a temperature sensor 156. The button 104 (FIG. 3) operates the switch 98 (see also FIG. 3) that is in electrical communication with the heating element 106 via the PCB 92. The indicator 152 (if provided) is in electrical communication with the PCB 92 and can be a light emitting diode visible from the exterior of the toothbrush sanitizer 10. The indicator 152 operates, i.e. illuminates, when the heating element 106 receives power from the power source. The indicator 152 can also provide an audible signal during stages of the sanitization process, e.g. start and finish. The timer 154 is in electrical communication with the PCB 92 and can be a solid state device, or other device, that controls the power delivery to the heating element 106. For example, the timer 154 can communicate through the controller 150 to the heating element 106 to stop the delivery of power to the heat source after a predetermined temperature has been measured for a predetermined amount of time. For example, the product sanitization process can be approximately 15 minutes at 150° F. Other times and temperatures can be programmed as well.

The temperature sensor 156 is also in electrical communication with the PCB 92 and can also communicate through the controller 150 to the heating element 106 to hold the heat within the baskets between a desired temperature range. For example, the maximum temperature can be approximately 185° F. and the controller can control the amount of power delivered to the heating element 106 as a function of the temperature that is sensed by the temperature sensor.

The toothbrush sanitizer has been described with reference to a particular embodiment. Modifications and alterations will occur to those upon reading the detailed description and examining the accompanying figures. The invention is not limited to only those embodiments and alternatives discussed above. Instead, the invention is broadly defined by the appended claims and the equivalents thereof.

1. A toothbrush sanitizer comprising:
at least one basket for holding an associated toothbrush or toothbrush head;
a housing that receives the at least one basket, the at least one basket being completely removable from the housing so that the at least one basket can be separately cleaned from the housing; and
a heating element connected with the housing for heating the associated toothbrush or toothbrush head.

2. The toothbrush sanitizer of claim 1, wherein the at least one basket includes a first basket and a second basket that is completely removable from the first basket, each basket being completely removable from the housing so that each basket can be separately cleaned from the housing.

3. The toothbrush sanitizer of claim 2, wherein the first basket is received in and supported by the second basket, and the first basket has a base that is vertically spaced above a base of the second basket when the first basket is received in the second basket.

4. The toothbrush sanitizer of claim 3, wherein the base of the first basket includes an opening that extends through the base.

5. The toothbrush sanitizer of claim 4, wherein the first basket includes internal walls that define a plurality of compartments, wherein the opening is formed in at least one of the compartments and has a diameter large enough to allow an associated electric toothbrush handle to fit through the opening.

6. The toothbrush sanitizer of claim 5, wherein each base includes a drain aperture, the drain aperture having a diameter smaller than the diameter of the opening.

7. The toothbrush sanitizer of claim 3, wherein the base of the first basket is spaced below the heating element between about 4.5 inches and about 5.5 inches.

8. The toothbrush sanitizer of claim 5, wherein the base of the second basket is spaced below the heating element between about 9 inches and about 10 inches.

9. The toothbrush sanitizer of claim 2, wherein the second basket is integrally formed with a front cover that cooperates with the housing to enclose an internal compartment that encloses the associated toothbrush or toothbrush head.

10. The toothbrush sanitizer of claim 9, wherein the first basket is disposed behind the front cover and within the internal compartment when the baskets are received in the housing.

11. The toothbrush sanitizer of claim 1, wherein the at least one basket is integrally formed with a front cover that cooperates with the housing to define an internal compartment that encloses the entire associated toothbrush or toothbrush head.

12. The toothbrush sanitizer of claim 1, wherein the heating element is a light bulb.

13. The toothbrush sanitizer of claim 12, further comprising a reflector for directing radiant energy from the light bulb toward the toothbrush.

14. The toothbrush sanitizer of claim 13, further comprising titanium dioxide disposed on the reflector.

15. The toothbrush sanitizer of claim 14, further comprising a UV filter disposed below the light bulb.

16. A method for sanitizing a toothbrush, the method comprising:
positioning a toothbrush in a lower basket so the toothbrush is in a substantially vertical orientation with bristles of the toothbrush being situated highest;
inserting the lower basket into a housing that contains an electrically powered heating element; and
operating a switch to deliver electricity to the heating element to heat the bristles of the toothbrush.

17. The method of claim 16, further comprising positioning a toothbrush head or a toothbrush in an upper basket in a substantially vertical orientation with bristles being situated highest, and inserting the upper basket into the lower basket prior to inserting the lower basket into the housing.

18. A toothbrush sanitizer comprising:
a first toothbrush basket;
a second toothbrush basket, the second toothbrush basket receiving the first toothbrush basket;
a housing that receives the toothbrush baskets when the second toothbrush basket is received in the first toothbrush basket; and
a heating element connected with the housing.

19. The toothbrush sanitizer of claim 18, wherein the toothbrush baskets are completely removable from the housing so that the toothbrush baskets can be cleaned separately from the housing.

20. The toothbrush sanitizer of claim 19, wherein the second toothbrush basket is integrally formed with a front cover that fits substantially flush with an outer surface of the housing to enclose an internal compartment, and the first toothbrush basket is disposed behind the front cover and within the internal compartment when the toothbrush baskets are received in the housing.
